(19) United States
(12) Patent Application Publication
(10) Pub. No.: US 2003/0154031 A1
(43) Pub. Date: Aug. 14, 2003

(54) Method and apparatus for the rapid evaluation of a plurality of materials or samples

(75) Inventors: Radislav Alexandrovich Potyrailo, Niskayuna, NY (US); William Guy Morris, Rexford, NY (US); Denyce Kramer Wicht, Saratoga Springs, NY (US)

Correspondence Address: GENERAL ELECTRIC COMPANY, GLOBAL RESEARCH CENTER, PATENT DOCKET RM. 4A59, PO BOX 8, BLDG. K-1 ROSS, NISKAYUNA, NY, 12309, US

(73) Assignee: General Electric Company

(21) Appl. No.: 10/074,544

(22) Filed: Feb. 14, 2002

(51) Int. Cl. G06F 19/00; G01N 33/48; G01N 33/50; C12M 1/34

(52) U.S. Cl. 702/19; 435/287.1

(57) ABSTRACT

An apparatus for the rapid evaluation of a plurality of materials or samples including a plurality of crystals operable for receiving an oscillating potential and oscillating, the plurality of crystals arranged in an array. The apparatus also including a plurality of oscillation devices operable for generating the oscillating potential, the plurality of oscillation devices arranged in an array. The apparatus further including means for measuring an output parameter each of the plurality of crystals. The plurality of crystals are remotely coupled to the plurality of oscillation devices such that the plurality of crystals are exposed to a first operating environment and the plurality of oscillation devices are exposed to a second operating environment. A method for enhancing the stability and the selectivity of each of a plurality of sensors of an array of sensors including modulating each of the plurality of sensors of the array of sensors with respect to a predetermined parameter. Each of the plurality of sensors of the array of sensors including a material that is sensitive to a given environment such that when the sensor is exposed to the given environment a property of the material will change, measurably changing an output parameter of the sensor. The degree of change in the output parameter is correlated to the degree to which the given environment is present. The apparatus and method of the present invention finding applicability in a variety of combinatorial chemistry applications.

### BACKGROUND OF THE INVENTION

The present invention relates generally to a method and an apparatus for the rapid evaluation of a plurality of materials or samples. More specifically, the present invention relates to a method and an apparatus for the rapid, simultaneous evaluation of a plurality of materials or samples utilizing an array of sensors remotely coupled to a plurality of signal processing electronics units.

In combinatorial chemistry applications, it is often desirable to rapidly evaluate a plurality of materials or samples. Preferably, these evaluations are carried out simultaneously. For example, it may be desirable to characterize and quantify the effect of a plurality of chemicals on a plurality of materials or coatings, i.e. the chemical resistance of the materials or coatings. It may also be desirable to detect the presence of a fluid of interest in a mixture of fluids, or to evaluate the mixture of fluids itself. It may further be desirable to study the extractability of different types of materials or additives from a plurality of samples.

Conventional methods for the measurement of chemical resistance typically require the exposure of a material sample to a solvent for extended periods of time. For example, such tests may last several days and utilize elevated temperatures. Thus, such methods are typically too time and resource consuming to be useful in the rapid screening of a large number of materials or samples, such as those developed in combinatorial chemistry applications. Likewise, the methods are difficult to apply to the measurement of multiple, small samples simultaneously. In addition, the accuracy of such measurements is typically limited by the ability to measure small mass gains and losses, thereby requiring large samples to detect mass changes.

Conventional methods for detecting material properties of interest, such as transition temperatures, storage modulus, loss modulus, absorption, plasticization, diffusion, permeation, physisorption, chemisorption, polymerization, corrosion, and the like, using sensors typically require that the measurement equipment be exposed to a variety of temperature, pressure, and chemical environments, many of which may be damaging to the delicate measurement equipment. Such methods are also limited by the stability and sensitivity of the sensors utilized.

Conventional methods for the study of the extractability of materials such as polyolefins, silicones, polycarbonates, polycarbonate blends, polycarbonate-polyorganosiloxane copolymers, polyetherimide resins, and the like also require the exposure of a large sample area or volume to a solvent for extended periods of time, and such analyses are typically not performed in a high-throughput mode. This extractability may be studied using, for example, water, different fuels, alkaline and acidic solutions, organic solvents of different polarities, and other solvent mixtures. Again, conventional extractability measurement methods are too time consuming to be useful in the rapid screening of a large number of materials, such as those developed in combinatorial chemistry applications. These methods are also difficult to apply to the measurement of multiple, small samples simultaneously.

Thus, what is needed is an apparatus for the rapid, simultaneous evaluation of a plurality of materials or samples in diverse application environments. The apparatus may preferably be used in a variety of applications and utilizes an array of sensors remotely coupled to a plurality of electronics units, such that the sensors may be subjected to variable environmental conditions, which may include variations in temperature, pressure, chemical composition, electromagnetic radiation level, and the like, without damaging the delicate oscillators and electronics. These sensors must be stable and selective if they are to be utilized to quantify volatile fluid compounds in real-world environments, especially over multiple measurement cycles. These fluid compounds may include gaseous or liquid components. With respect to the measurement of extractables, what is also needed is a method for the rapid evaluation of the extractability of materials from a plurality of samples capable of increased measurement speed, obtaining parallel measurements, and analyzing picogram quantities of dissolved residual materials in solutions.

### BRIEF SUMMARY OF THE INVENTION

The present invention provides an apparatus for the rapid, simultaneous evaluation of a plurality of materials or samples that utilizes an array of sensors remotely coupled to a plurality of electronics units, such that the sensors may be subjected to a variety of temperature, pressure, chemical, and electromagnetic radiation environments without damaging the delicate electronics units. In other words, a first operating environment may be created for the system components and a second operating environment may be created for the plurality of oscillation devices. These sensors are stable and selective such that they may be utilized to characterize and quantify fluid compounds in real-world environments, especially over multiple measurement cycles. The apparatus of the present invention may be utilized in a variety of applications including, but not limited to, the study of the chemical resistance, radiation resistance, and hydrolytic resistance of materials or coatings, the detection of fluids and the evaluation of mixtures of fluids, and the measurement of extractables.

The present invention also provides a method for the rapid, simultaneous evaluation of the extractability of materials from a plurality of samples capable of increased measurement speed, obtaining parallel measurements, and analyzing picogram quantities of dissolved residual materials in solutions.

Other applications of the apparatus of the present invention include the evaluation of material properties such as transition temperatures, storage modulus, loss modulus, absorption, plasticization, diffusion, permeation, physisorption, chemisorption, polymerization, corrosion, and the like.

The present invention involves and utilizes sensors based on thickness-shear mode (TSM) devices. The surface of a crystal of each of the sensors is coated with a material that is susceptible to weight or viscoelastic property changes when exposed to various chemical species and ambient environments. For example, a mixture of several fluids may be analyzed utilizing an array of TSMs, each of which is coated with a material displaying a predetermined sensitivity to an individual fluid in the mixture. This apparatus necessitates the use of an electronic circuit that is capable of accurately detecting small changes in resonant frequency within a short time period for each TSM. The apparatus may be used to detect the presence of a fluid of interest in the mixture of fluids, or to generally characterize the mixture of fluids itself.

In one embodiment of the present invention, an apparatus for the rapid evaluation of a plurality of materials includes a plurality of crystals operable for receiving an oscillating potential and oscillating, the plurality of crystals arranged in an array. The apparatus also includes a plurality of oscillation devices operable for generating the oscillating potential, the plurality of oscillation devices arranged in an array. The apparatus further includes means for measuring an output parameter each of the plurality of crystals. The plurality of crystals are remotely coupled to the plurality of oscillation devices such that the plurality of crystals are exposed to a first operating environment and the plurality of oscillation devices are exposed to a second operating environment. The process is controlled automatically by an electronic device, such as a computer, to provide desired environmental parameters during the evaluation of materials.

In another embodiment of the present invention, a method for enhancing the stability and the selectivity of each of a plurality of sensors of an array of sensors includes modulating each of the plurality of sensors of the array of sensors with respect to a predetermined parameter. Each of the plurality of sensors of the array of sensors includes a material that is sensitive to a given environment such that when the sensor is exposed to the given environment a property of the material will change, measurably changing an output parameter of the sensor. The degree of change in the output parameter is correlated to the degree to which the given environment is present. The degree of change itself is modulated by the change in the modulation period, modulation duty cycle, modulation waveform, modulation depth, modulation phase between several modulated parameters, and the like.

In a further embodiment of the present invention, a method for the rapid evaluation of the extractability of materials from a plurality of samples includes providing a plurality of samples and disposing the plurality of samples in a plurality of solvents for a first predetermined period of time. The method also includes providing a plurality of acoustic wave devices and measuring a predetermined output parameter of each of the plurality of acoustic wave devices. The method further includes disposing the plurality of acoustic wave devices in the plurality of solvents for a second predetermined period of time, removing the plurality of acoustic wave devices from the plurality of solvents, and measuring the predetermined output parameter of each of the plurality of acoustic wave devices. The method further includes correlating the change in the predetermined output parameter of each of the plurality of acoustic wave devices to the extractability of materials from the plurality of samples.

In a further embodiment of the present invention, an apparatus for the rapid evaluation of the extractability of materials from a plurality of samples includes a plurality of wells, the plurality of wells arranged in an array. The apparatus also includes a plurality of samples and a plurality of solvents disposed within the plurality of wells, the plurality of samples and the plurality of solvents combining to form a plurality of solutions containing the materials. The apparatus further includes a plurality of acoustic wave devices, wherein each of the plurality of acoustic wave devices includes at least one surface operable for attracting at least one of the materials from the plurality of solutions, the plurality of acoustic wave devices arranged in an array. The apparatus further includes means for measuring a predetermined output parameter of each of the plurality of acoustic wave devices and a correlation factor operable for correlating a change in the predetermined output parameter of each of the plurality of acoustic wave devices to the extractability of the materials from the plurality of samples.

In a further embodiment of the present invention, an apparatus for the rapid evaluation of chemical sensitivity properties of a plurality of materials includes a plurality of crystals coated with materials of interest and operable for receiving an oscillating potential and oscillating, the plurality of crystals arranged in an array. The apparatus also includes a plurality of oscillation devices operable for generating the oscillating potential, the plurality of oscillation devices arranged in an array. The apparatus further includes means for measuring an output parameter of each of the plurality of crystals. The plurality of crystals is remotely coupled to the plurality of oscillation devices such that the plurality of crystals are exposed to a first operating environment and the plurality of oscillation devices are exposed to a second operating environment. The evaluation process of the chemical sensitivity properties is controlled automatically by an electronic device, such as a computer, to provide desired concentrations of fluids delivered to the array of coated crystals. A material coated onto an acoustic wave device has a chemical sensitivity property if, upon exposure of the coated device to an environment containing a fluid of interest, the device exhibits a measurable and reproducible change in its output parameter.

### BRIEF DESCRIPTION OF THE DRAWINGS

FIG. 1 is a perspective view of one embodiment of the apparatus for the rapid evaluation of the extractability of materials from a plurality of samples;

FIG. 2 is a flow chart of one embodiment of the method for the rapid evaluation of the extractability of materials from a plurality of samples;

FIG. 3 is a schematic view of one embodiment of the apparatus for the rapid measurement of an output parameter of a plurality of acoustic wave devices;

FIG. 4 is a schematic view of signal generation and collection associated with one embodiment of the apparatus for the rapid measurement of an output parameter of a plurality of acoustic wave devices;

FIG. 5 is a flow chart of one embodiment of a method for utilizing the apparatus for the rapid measurement of an output parameter of a plurality of acoustic wave devices;

FIG. 6 illustrates a modulation sequence of analyte concentration with a variable modulation cycle time;

FIG. 7 illustrates the dynamic response of a sensor coated with a Teflon AF film upon exposure to a sinusoidally-modulated concentration of toluene vapor;

FIG. 8 illustrates the response of a sensor coated with a Teflon AF film upon periodic exposure to toluene vapor over the concentration range from about 0 to about 105 ppm with the exposure pattern shown in FIG. 7;

FIG. 9 illustrates the dynamic response of a sensor coated with a poly(vinyl propionate) film upon exposure to a sinusoidally-inodulated concentration of toluene vapor;

FIG. 10 illustrates the response of a sensor coated with a poly(vinyl propionate) film upon periodic exposure to toluene vapor over the concentration range from about 0 to about 105 ppm with the exposure pattern shown in FIG. 9;

FIG. 11 illustrates the dynamic response of a sensor coated with a Siltem 2000 film upon exposure to a sinusoidally-modulated concentration of TCE vapor with a variable modulation cycle time;

FIG. 12 illustrates the response of a sensor coated with a Siltem 2000 film upon periodic exposure to TCE vapor over the concentration range from about 0 to about 110 ppm with the exposure pattern shown in FIG. 11;

FIG. 13 illustrates the dynamic response of a sensor coated with an Ultem film upon exposure to a sinusoidally-modulated concentration of TCE vapor with a variable modulation cycle time;

FIG. 14 illustrates the response of a sensor coated with an Ultem film upon periodic exposure to TCE vapor over the concentration range from about 0 to about 110 ppm with the exposure pattern shown in FIG. 13;

FIG. 15 illustrates a typical response of a 24-channel system to short pulses of carbon tetrachloride vapor, water vapor, and TCE vapor; and

FIG. 16 illustrates the response of the 24-channel system to 100 ppm of toluene vapor.

### DETAILED DESCRIPTION OF THE INVENTION

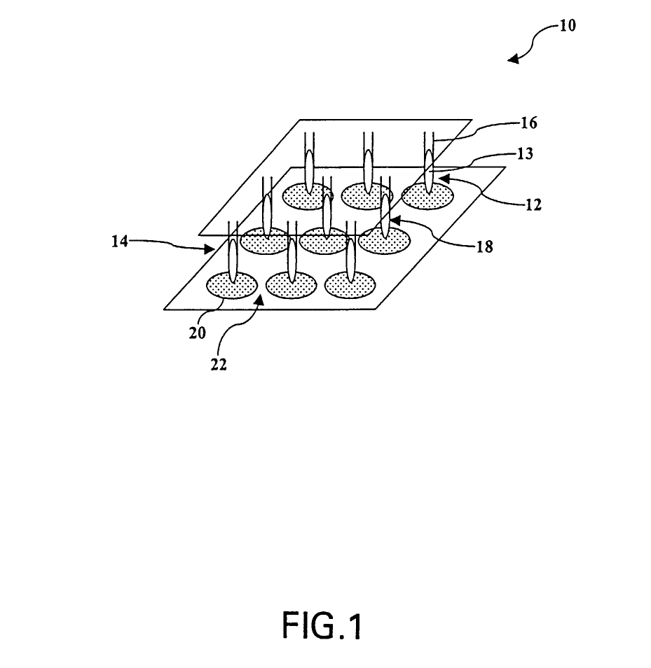

Referring to FIG. 1, in one embodiment of the present invention, the apparatus 10 for the rapid evaluation of a plurality of materials or samples includes a plurality of acoustic wave devices 12 arranged in an array 14. Each acoustic wave device 12 includes a piezoelectric transducer, such as a thickness-shear mode (TSM) device, coupled to a pair of opposed electrodes 16. The piezoelectric crystal 13 of each acoustic wave device 12 oscillates with a predetermined fundamental frequency when an oscillating potential is applied to the electrodes 16 that may be measured using a conventional frequency counter of a type well known to those of ordinary skill in the art. This fundamental oscillation frequency varies as a function of the mass of the acoustic wave device 12. Thus, for example, minute quantities of material extracted from a sample, dissolved in a solvent, and deposited onto a surface 18 of the acoustic wave device 12 after solvent evaporation or removal of the acoustic wave device 12 from the solvent may be detected by measuring a change in the fundamental oscillation frequency of the acoustic wave device 12. The fundamental oscillation frequency of the acoustic wave device 12 may also vary as a function of a viscoelastic property of a coating or material deposited on its surface 18. Other output parameters of the acoustic wave device 12, and the changes therein, may also be studied.

The change in mass of an oscillating crystal 13 of a TSM device is determined utilizing the change in the resonant frequency of the crystal according to the Sauerbrey equation:

Δf=−2fₒ²(m/A)(μ_{Q}ρ_{Q})^{-1/2}, (1)

where Δf is the change in the resonant frequency of the crystal 13 upon material deposition or removal, fₒ is the fundamental resonant frequency of the unloaded device, μ_{Q} is the shear modulus of the piezoelectric substrate, ρ_{Q} is the substrate density, m is the total mass of material deposited from or removed to solution from both faces of the crystal 13, and A is the active surface area of one face of the crystal 13. Exemplary piezoelectric transducers include 10 MHz AT-cut quartz crystals with an active electrode area of about 0.2 cm². The mass sensitivity of a crystal 13 per unit frequency change is:

m/Δf=−A/(2fₒ²)(μ_{Q}ρ_{Q})^{1/2}. (2)

The minus sign indicates that upon an increase in mass, the oscillation frequency decreases, while upon a decrease in mass, the oscillation frequency increases. For the AT-cut quartz crystal, μ_{Q} may be about 2.947×10¹¹ g/(cm s²) and ρ_{Q} may be about 2.648 g/cm³. The mass sensitivity of the device is:

m/Δf=−0.883×10⁻⁹ g/Hz. (3)

The above relationship demonstrates that the acoustic wave device 12 may be used to detect a mass change of 0.883 ng when the resolution of frequency measurement is 1 Hz, the typical noise level of a frequency measurement. This mass sensitivity may be improved, however, if an adequate gate time and temperature stabilization are utilized during frequency measurement, as is well known to those of ordinary skill in the art. In such cases, the noise in frequency measurements may be reduced to about 0.05 Hz, providing a mass resolution of about 44 pg.

Utilizing equation (1), the change in resonant frequency, Δf_{F}, may be related to the thickness of a film deposited onto the surface 18 of the crystal 13 via the following equation:

Δf_{F}=−4fₒ²ρ_{F}d_{F}(μ_{Q}ρ_{Q})^{-1/2}, (4)

where ρ_{F} and d_{F} are, respectively, the density and thickness of the film. For the AT-cut quartz crystal oscillating at about 10 MHz, the film thickness is given by:

d_{F}=2.2×10⁹Δf_{F}/ρ_{F}. (5)

Other types of piezoelectric transducers may be included in the apparatus or to carry out the methods of the present invention. For the measurement of extractables, the apparatus of the present invention may also include a plurality of wells 20, arranged in an array 22, suitable for containing a plurality of solvents and/or samples. Further, a conventional liquid-handling instrument, such as a Quadra 96 Model 230 Liquid Delivery System (Tomtec, Orange, Conn.) or an eight-probe liquid-handling system, may be utilized to remove samples from the wells and to deposit the samples directly or indirectly onto the crystals 13 of the array 14 of acoustic wave devices 12.

As described above, an acoustic wave transducer includes a piezoelectric crystal that allows transduction between electrical and acoustic energies. These transducers are known to those of ordinary skill in the art in a number of configurations. These configurations may be described based upon their unique acoustic modes, such as thickness-shear mode (TSM), surface acoustic wave (SAW), acoustic plate mode (APM), flexural plate wave (FPW), and surface transverse wave (STW). Non-piezoelectric acoustic wave transducers may be also utilized. A thin-rod acoustic wave (TRAW) device is an example of such a non-piezoelectric acoustic wave transducer. The TRAW device may be operated at relatively low frequencies (˜200 kHz). Other acoustic wave transducers may also be made of non-piezoelectric materials. These devices include, for example, cantilevers, torsion resonators, tuning forks, unimorphs (i.e. a type of single-pronged tuning fork), bimorphs (i.e. a type of two-pronged tuning fork), membrane resonators, etc. Such devices may be made of metal, glass, etc.

The operating frequencies of these acoustic wave transducers may be in the following approximate ranges: TSM, 0.1-70 MHz; SAW, 30-10,000 MHz; APM, 20-500 MHz; FPW, 0.01-10 MHz; STW, 100-1,000 MHz; and TRAW, 0.2-1 MHz. For other non-piezoelectric acoustic-wave devices, such as the cantilevers, torsion resonators, tuning forks, unimorphs, bimorphs, membrane resonators, etc., the operating frequencies are in the range of about 1 Hz-1 MHz. In general, the acoustic wave transducers of the present invention operate in a frequency range of about 10 GHz-0.1 Hz, preferably in the range of about 500 MHz-1 kHz, and more preferably in the range of about 100 MHz-100 KHz. The active surface area of the acoustic wave transducers of the present invention are in the range of about 1×10⁻⁶ cm²-2 cm², preferably in the range of about 1×10⁻⁵ cm²-5×10⁻¹ cm², and more preferably in the range of about 1×10⁻⁴ cm²-5×10⁻² cm². The acoustic wave transducers are typically about 0.2 mm-50 mm in size and have cylindrical or rectangular shapes. The acoustic wave transducers may be about 10 microns-2 mm in thickness, although other shapes and sizes may be utilized. The minute quantity of material deposited on each acoustic wave transducer and used for evaluation may be in the range of about 1 picogram-1 milligram, preferably in the range of about 100 picogram-10 milligram, and more preferably in the range of about 1 nanogram-1 microgram. The quantity of the material is dependent upon the operating frequency of the transducer.

The acoustic wave transducers may be one or two-port devices. In one-port devices, such as TSM devices, a single port serves as both an input and an output. The input signal excites an acoustic mode, which in turn generates charges on the input electrode. These signals combine to produce an impedance variation that constitutes the TSM device response. In two-port devices, one port is used as the input port and the another port is used as the output port. The input signal generates an acoustic wave that propagates to a receiving transducer which generates a signal on the output port. The relative signal level and phase delay between the input port and the output port constitute two responses.

In a TSM device, an oscillating potential is applied to two electrodes deposited onto and in contact with opposite sides of an acoustic wave transducer, such as a quartz crystal. This acoustic wave transducer oscillates in, for example, the thickness-shear mode, with a fundamental resonant frequency measured using a conventional frequency counter or the like. Such an acoustic wave device allows measurement of such variations as the change in mass of a material or coating applied to the oscillating crystal, as well as several other properties of the material such as density, crystallinity, and viscosity, after accounting for other factors, such as the dimensions and other parameters of the crystal, as well as variables, such as the temperature at which measurement is made.

The materials of acoustic wave transducers include a substrate such as a quartz, lithium niobate, nitride, lithium tantalate, bismuth germanium oxide, aluminum nitride, or gallium arsenide substrate, and acoustic wave films (ZnO and AlN). Non-piezoelectric materials may also be used. The measurement of the acoustic wave transducer properties is made using electronic equipment, such as a network analyzer, a vector voltmeter, an impedance analyzer, a frequency counter, a phase interferometer, and an in-phase and quadrature demodulator.

The frequency of the oscillating crystal may be measured using a time interval analyzer instrument, rather than simply a conventional counter-timer instrument. The counter-timer instrument typically has a measurement uncertainty of about 1 count for the time interval chosen for measurement, i.e. about 1 Hz uncertainty for a 1 second measurement, about 10 Hz uncertainty for a 100 millisecond measurement, etc. The time interval analyzer instrument (such as Model GT654, Guide Technology Incorporated, Sunnyvale, Calif.) has an interval measurement accuracy of about 75 picoseconds. Thus, the time interval between the rising edge of the 1^{st} and the 1,000,000^{th} pulses of a 10 MHz oscillator (100 milliseconds) may be measured to about 75 picoseconds, or about 750 parts per trillion. This is equivalent to a frequency measurement uncertainty of about 0.0075 Hz for the time interval analyzer instrument, as compared to about 10 Hz for the counter-timer instrument.

It is important to note that, in various applications, a material, coating, or film may be applied to the acoustic wave devices 12 using thin-film deposition techniques in combination with physical masking techniques or photolithographic techniques. Such thin-film deposition techniques may generally be broken down into the following four categories: evaporative methods, glow discharge processes, gas-phase chemical processes, and liquid-phase chemical techniques. Included within these four categories are, for example, sputtering techniques, spraying techniques, laser ablation techniques, electron beam or thermal evaporation techniques, ion implantation or doping techniques, chemical vapor deposition techniques, as well as other techniques used in the fabrication of integrated circuits. All of these techniques may be applied to deposit highly uniform layers, i.e., thin-films, of the various materials of interest onto selected regions of each acoustic wave device 12. Other types of coating procedures are also applicable in conjunction with the methods of the present invention to deposit the materials of interest. These other coating procedures may include, for example, spin-coating, brushing, and laser deposition. Conventional liquid-handling instruments (for example, Quadra 96 Model 230 liquid delivery system, Tomtec, Orange, Conn., and an eight-probe liquid handler system from Gilson) may be utilized to deposit solutions of coatings onto the individual transducers of an array of acoustic wave devices 12. When the transducer array and solvent well array have different layouts, a conventional liquid-handling instrument with variable spacing between the liquid-delivery tips (for example, Model Lissy, Zinsser Analytic, Frankfurt, Germany) is utilized to transfer solvents from the wells directly onto the array of crystals or into another array of wells.

The delivery of solid components of coating formulations to solvent-containing wells is accomplished using, for example, a conventional solid-handling instrument. Such an instrument is capable of delivering a predetermined amount of a solid material into each of the wells, previously filled with a solvent, or filled with a solvent after the delivery of a solid sample. Stirring, if needed, is provided using, for example, known stirring equipment for multiple wells (e.g., a heating/stirring module such as Reacti-Therm III, Pierce, Rockford, Ill.). Other alternative methods for transferring a portion or all of the solvents or other chemicals in the wells to the acoustic wave devices are well known to those of ordinary skill in the art.

Optionally, the material or combination of materials deposited onto each acoustic wave transducer may form a coating having a plurality of layers, where the coating may be a multi-functional coating having an overall function dictated by a predefined functional role of each layer. The plurality of materials may be combined such that multiple organic materials are combined into a coating. By providing these various combinations of the plurality of materials, the interaction and compatibility of various combinations may be determined through the use of the testing device. The coating is a material or a combination of materials deposited on the surface of the substrate. These materials may remain as separate homogenous materials, or they may interact, react, diffuse, mix, or otherwise combine to form a new homogeneous material, a mixture, a composite, or a blend. Each member of the array of coatings is distinguishable from the others based upon its location. Further, each member of the array of coatings may be processed under the same conditions and analyzed to determine its performance relative to functional or useful properties, and then compared with each of the other members of the array of coatings to determine its relative utility. Alternatively, each member of the array of coatings may be processed under different conditions and the processing methods may be analyzed to determine their performance relative to functional or useful properties, and then compared with each other to determine their relative utility.

A curing source may also be utilized and is a device in communication with each of the plurality of materials causing a reaction or solvent evaporation with one or a combination of the plurality of materials. For example, the reaction may be a polymerization reaction, a cross-linking reaction, a small molecule reaction, an inorganic phase reaction, and other similar reactions appropriate for the delivered material(s). The curing source accomplishes this by delivering a curing medium. The curing medium may be any form of energy or suitable material that interacts with the combination of the plurality of materials forming the coating to sufficiently cure the coating. Suitable examples of curing environments preferably include those created by a curing source selected from the group including ultraviolet (UV) radiation, infrared (IR) radiation, thermal radiation, microwave radiation, visible radiation, narrow-wavelength radiation, laser light, and humidity. The coating may also be conditioned. Conditioning may include cross-linking, solvent evaporation, weathering, exposure to heat, UV-visible radiation, electromagnetic radiation, laser light, and the like.

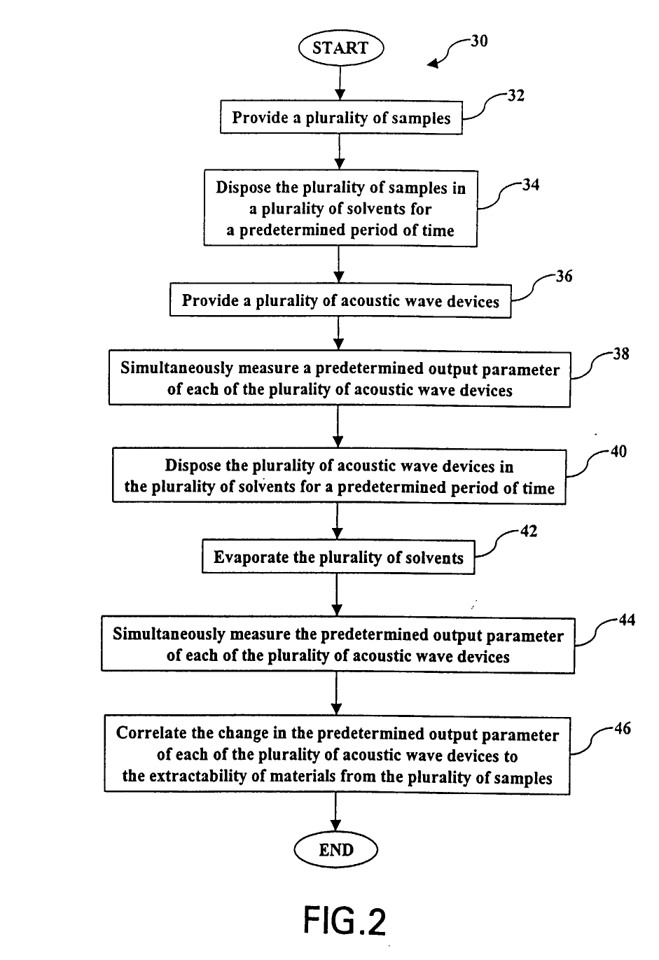

Referring to FIG. 2, in one exemplary application of the apparatus of the present invention, a method 30 for the rapid evaluation of the extractability of materials from a plurality of samples includes providing a plurality of samples (Block 32) and disposing the plurality of samples in a plurality of solvents for a predetermined period of time (Block 34). As described above, the extractability of different types of chemical components and additives from a variety of materials may be of interest. For example, the extractability of additives, oligomers, and any other low-molecular weight components in polyolefins, silicones, polycarbonates, polycarbonate blends, polycarbonate-polyorganosiloxane copolymers, polyetherimide resins, and the like may be of interest. This extractability may be studied using, for example, water, different fuels, alkaline and acidic solutions, organic solvents of different polarities, and other solvent mixtures. The method 30 also includes providing a plurality of acoustic wave devices (Block 36). As discussed above, each acoustic wave device includes a piezoelectric transducer, such as a TSM device. A predetermined output parameter, such as oscillation frequency, is simultaneously measured for each of the plurality of acoustic wave devices (Block 38). The plurality of acoustic wave devices are then disposed in the plurality of solvents, presumably including extractables from the plurality of samples, for a predetermined period of time (Block 40), the plurality of solvents are allowed to evaporate (Block 42), and the predetermined output parameter is again simultaneously measured for each of the plurality of acoustic wave devices (Block 44). Alternatively, the plurality of acoustic wave devices may simply be removed from the plurality of solvents prior to the measurement of the predetermined output parameter. Finally, the change in the predetermined output parameter of each of the plurality of acoustic wave devices is correlated to the extractablity of materials from the plurality of samples (Block 46).

### WORKING EXAMPLE 1

In one working example of the present invention, the measurement of extractables from cured silicones was utilized to demonstrate the applicability of the techniques discussed above. A 50 μL volume of silicone formulation was deposited as a thin film in a scintillator vial. The concentrations of a Pt catalyst used were about 0 ppm, about 50 ppm, and about 200 ppm. The films were cured at about 80 degrees C. for about 30 min. Extraction was performed utilizing methyl ethyl ketone (MEK) (600 μL). The samples were exposed to the solvent overnight. For measurement, 20 μL of solution was applied to each transducer. The TSM transducer (10-MHz AT-cut quartz crystal) response ranges are illustrated below:

TABLE I
Transducer Response as a Function of Pt Concentration

| Pt Concentration (ppm) | Transducer Response (Hz) |
|---|---|
| 0 | 110-150 |
| 50 | 8-10 |
| 200 | 8-10 |

In another exemplary application of the apparatus of the present invention, the apparatus 10 (FIG. 1) may be utilized to detect the presence of a variety of temperature, pressure, and chemical environments. In such cases, the apparatus 10 includes a plurality of crystals 13 (FIG. 1) arranged in an array 14 (FIG. 1). The surface 18 (FIG. 1) of each of the plurality of crystals 13 is coated with a material that is sensitive to a given temperature, pressure, or chemical environment, forming a sensor. When the sensor is exposed to such an environment, the mass of the sensor or a viscoelastic property of the coating material will change, changing the fundamental oscillation frequency of the crystal 13. The degree of change in the fundamental oscillation frequency of the crystal 13 may be correlated to the degree to which the environment of interest is present, i.e. the strength or concentration of the environment.

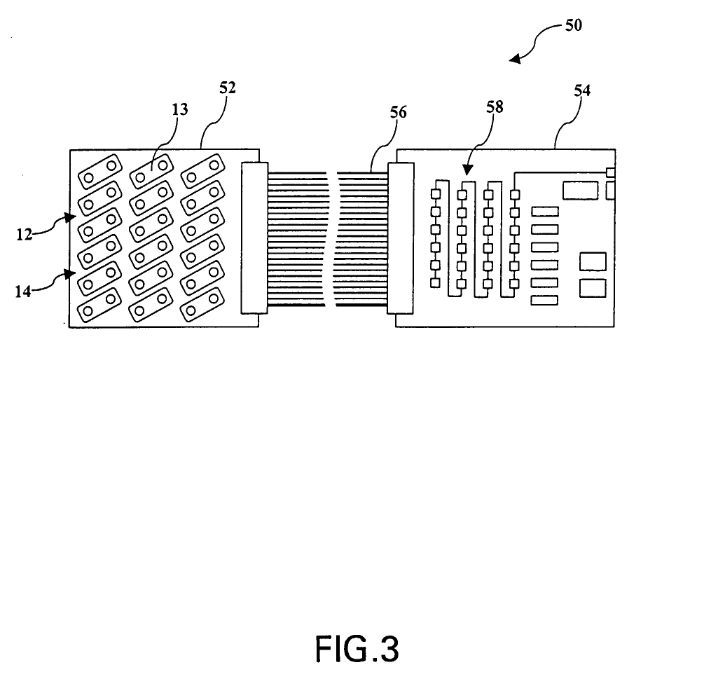

In any of the embodiments or exemplary applications discussed above, the array 14 of acoustic wave devices 12 (FIG. 1) or sensors must be measured independently, quickly, and accurately. Referring to FIG. 3, in another embodiment of the present invention, an apparatus 50 for the rapid measurement of an output parameter of a plurality of acoustic wave devices 12 includes a plurality of crystals 13 arranged in an array 14 and coupled to a first printed circuit board 52. The first printed circuit board 52 may accommodate, for example, 24 crystals 13 in a 4×6 array 14. The first printed circuit board 52 is coupled to a second printed circuit board 54 via a cable 56. A plurality of oscillation devices 58, such as integrated circuit oscillators, are coupled to the second printed circuit board 54. Each of the oscillation devices 58 emits a signal that is received, one signal at a time, by a TTL multiplexor integrated circuit connected to a time interval analyzer circuit card disposed within a personal computer. The personal computer also contains software operable for selecting one of a plurality of signal channels at a time and measuring it for a predetermined period of time, such as about 10 msec. Data collection software may be written in, for example, LabVIEW (National Instruments). The oscillators 58 making up the array of oscillators 58 are preferably addressed sequentially. For an oscillator 58 measured over a fixed time period of about 40 msec, about 24 oscillators may be measured in about 1 sec, each with an accuracy of about 0.019 Hz. The fundamental oscillation frequency of each of the plurality of crystals may be measured and stored in a table, which may be displayed on a computer monitor and downloaded into a database. A plurality of signals may be measured in, for example, about 500 msec and the standard deviation of ten fundamental oscillation frequency measurements for a 10 MHz signal from a single channel is typically about 0.1 Hz. It should be noted that, by remotely coupling the array 14 of acoustic wave devices 12 to the plurality of oscillation devices 58 and the electronics unit, the acoustic wave devices 12 may be subjected to a variety of temperature, pressure, chemical, and electromagnetic radiation environments without damaging the delicate oscillation devices 58 and electronics.

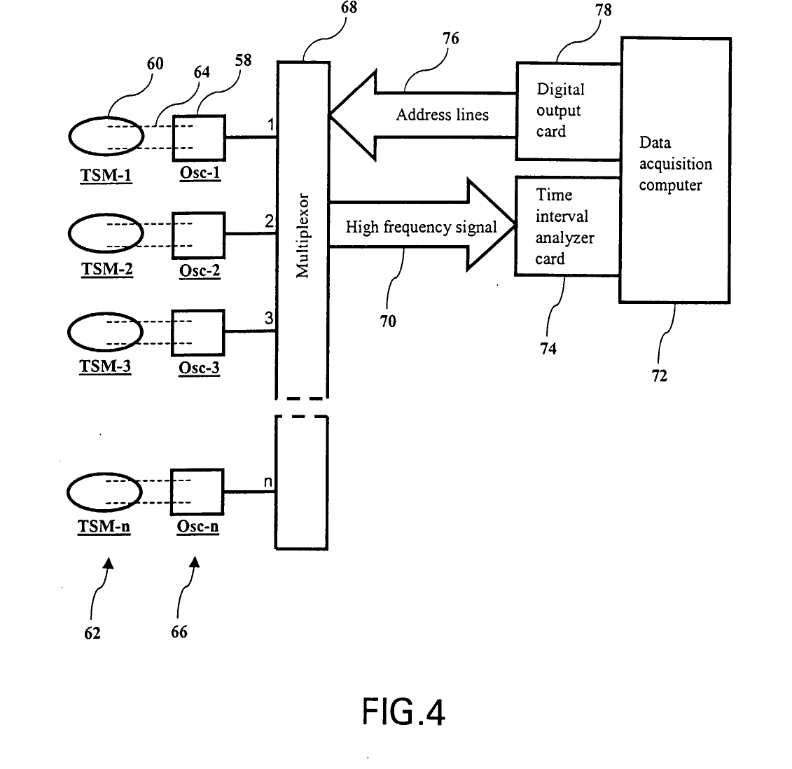

Referring to FIG. 4, in a further embodiment of the present invention, each TSM 60 forming an array of TSMs 62 is coupled, via the interconnecting cable 64, to an array of oscillators 66. Each oscillator 58 forming the array of oscillators 66 is coupled to the multiplexor 68. The multiplexor 68 sends a high frequency signal 70 to a data acquisition computer 72 via the time interval analyzer card 74. The multiplexor 68 also receives address lines 76 from the data acquisition computer 72 via a digital output card 78.

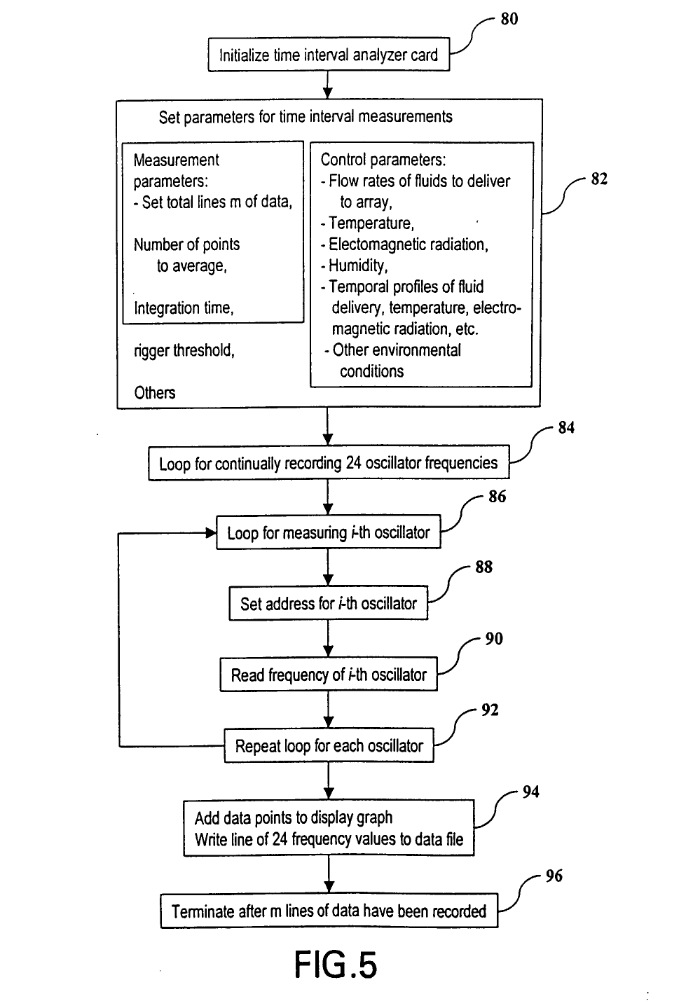

Referring to FIG. 5, in a further embodiment of the present invention, the methodology followed by the apparatus described above includes first initializing the time interval analyzer card 74 (FIG. 4) (Block 80). Next, the method includes setting the parameters for the time interval measurements and instrument control (Block 82). Measurement parameters include setting the total lines of data, setting the number of points to average, setting the trigger threshold, and setting the integration time. Instrument control parameters include flow rates of fluids delivered to the array of transducers, temperature, electromagnetic radiation, humidity, temporal profiles of fluid delivery, and temporal profiles of any other environmental condition that affects the response of the transducers. A plurality of loops are then established, including a loop for continually recording the 24 oscillator frequencies (Block 84) and a loop for measuring the i-th oscillator (Block 86). The address is then set for the i-th oscillator (Block 88), the frequency of the i-th oscillator is read (Block 90), and the loop is repeated for each oscillator (Block 92). Finally, the data points are added to the display graph and a line of 24 frequency values is written to the data file (Block 94), and the process is terminated after m lines of data have been recorded (Block 96).

As described above, sensors and sensor arrays must be stable and selective if they are to be utilized to quantify fluid compounds in real-world environments, especially over multiple measurement cycles. In particular, the high-throughput screening of combinatorially-developed materials requires exceptional stability and selectivity of sensors and sensor arrays. When low levels of analytes are utilized, stability and selectivity become key parameters that affect overall analysis success. Typical sorbing polymer coatings may cause a noticeable long-term drift in sensors and sensor arrays. This drift may result in the degradation of the detection limit and the useful life of the sensor or sensor array. Modulation of a variety of sensor parameters has been attempted by those of ordinary skill in the art in the past. For example, the periodic modulation of analyte concentration, ambient temperature, sensor temperature, redox potential, and measurement voltage has been attempted with step, ramp, saw, sinusoidal, and other functions, all with limited success.

The present invention provides a signal-generation method and apparatus for improving the stability and selectivity of sensors and sensor arrays. The effects of the variable sorption/desorption characteristics of different vapors upon interaction with coatings disposed on a sensor are enhanced. This enhancement is achieved through the modulation of the sample flow rate and/or sensor temperature. Preferably, a sinusoidal modulation method is utilized. In another embodiment of the present invention, a portable analyte-removal source, such as a corona discharge, is utilized in combination with a sensor. When coupled with the operation of the analyte-removal source, a gas stream reaching a given sensor has nullified or reduced contamination and is free from analyte vapors and analyte interference. This cleaned gas flow provides a baseline for sensor correction and self-calibration.

Enhancement is achieved through modulation of the sample flow rate, keeping the flow rate through a given sensor constant. Thus, the concentration of an analyte is modulated from its maximum to zero, providing a maximum modulation depth. Other modulation depths arc possible, for example, from the maximum concentration to a predetermined concentration. A variety of modulation schemes may be utilized, including sinusoidal modulation. The Fourier transformation (FT) of the recorded signal from a single sensor and the discrimination between different vapors is accomplished utilizing the FT signature of each vapor. Specifically, the signatures are analyzed utilizing the higher harmonics of the FT spectrum. Optionally, other modulation schemes (e.g. step, ramp, saw) may be utilized. The modulation of analyte concentration around each sensor element is preferable to the modulation of temperature relative to each sensor. Modulation over a wide-range of temperatures is needed for effective operation of a temperature modulation method. However, it may cause premature degradation of sensitive materials and, as a result, is less desirable than analyte modulation.

In another embodiment of the present invention, temperature modulation is utilized with a data analysis approach similar to that utilized for the modulation of analyte flow. This approach is preferable in those situations where an irreversible response or slow desorption of a given vapor is observable at normal operating conditions of a given sensor.

Signal processing algorithms for the analysis of a sinusoidally-modulated sensor signal may be adapted from those used for the analysis of phase-resolved fluorescence sensor signals, well known to those of ordinary skill in the art. Additional modulation parameters may also be introduced. Modulation parameters may include, but are not limited to, signal modulation depth, signal modulation delay, phase modulation, signal profile upon analyte sorption, and signal profile upon analyte desorption. In a phase modulation method, the several environmental parameters described above are applied with temporally variable phase of modulation between them. Signal profiles may be analyzed in the initial, intermediate, and final regions. Further, signal modulation may be performed with a variable pitch, or modulation cycle. By these methods, a single modulation pattern contains regions where the sensor has maximum sensitivity and selectivity.

In a further embodiment of the present invention, a portable corona discharge source is utilized in combination with a given sensor. The portable corona discharge source preferably operates from a battery and comprise a modular attachment to the sensor unit. Upon operation of the portable corona discharge source, a gas stream reaching the sensor has eliminated or reduced contamination. Such a cleaned gas flow provides a baseline for sensor correction and self-calibration. Other vapor purification sources and/or rough separation devices, such as those used in chromatography and air purification, may be utilized. For example, a Nafion tube may be utilized to reduce the moisture content of an analyzed vapor mixture.

### WORKING EXAMPLE 2

In another working example of the present invention, a thin film of a polymer material was applied to the surface of a TSM. The change in polymer mass was monitored upon exposure to analyte vapor. An AT-cut quartz crystal with gold electrodes was utilized as the sensor substrate. The crystals oscillated in the thickness-shear mode with a fundamental frequency of about 10 MHz. Film deposition was accomplished utilizing Teflon AF, poly(vinyl propionate), Ultem, and Siltem 2000 polymers dissolved in suitable solvents. Use of Teflon AF, Ultem, and Siltem 2000 sensor materials is the subject of other patent applications (EP1076238 and WO0167086). The polymer solutions were applied to both sides of the crystal and dried. The coated crystals were arranged in a low-dead volume flow-through gas cell. The resonant oscillation frequency of the crystals was monitored as a function of analyte concentration in a gas mixture. Various vapor concentrations were generated utilizing gas tanks and diluting the gases therein with dry nitrogen. The gas flow rate was kept constant at about 480 cm³/min using mass-flow controllers. Measurements were performed at about 20 degrees C.

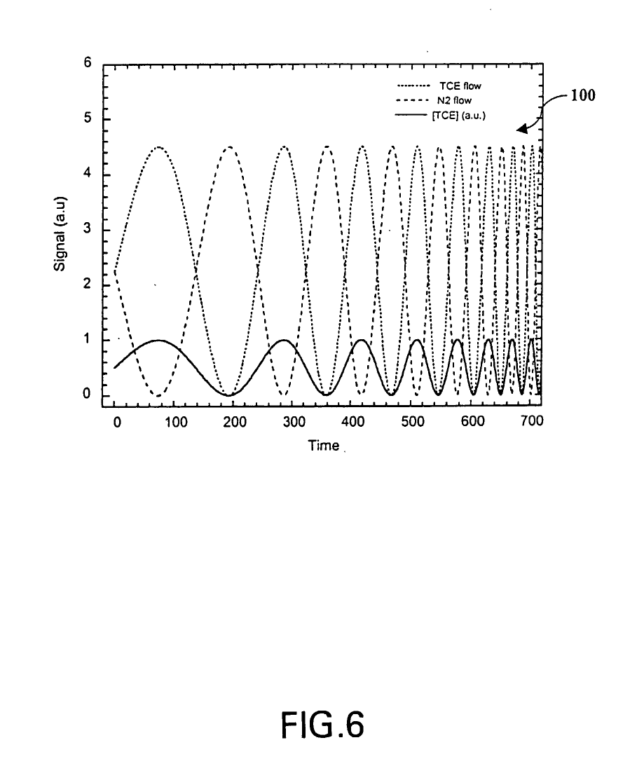

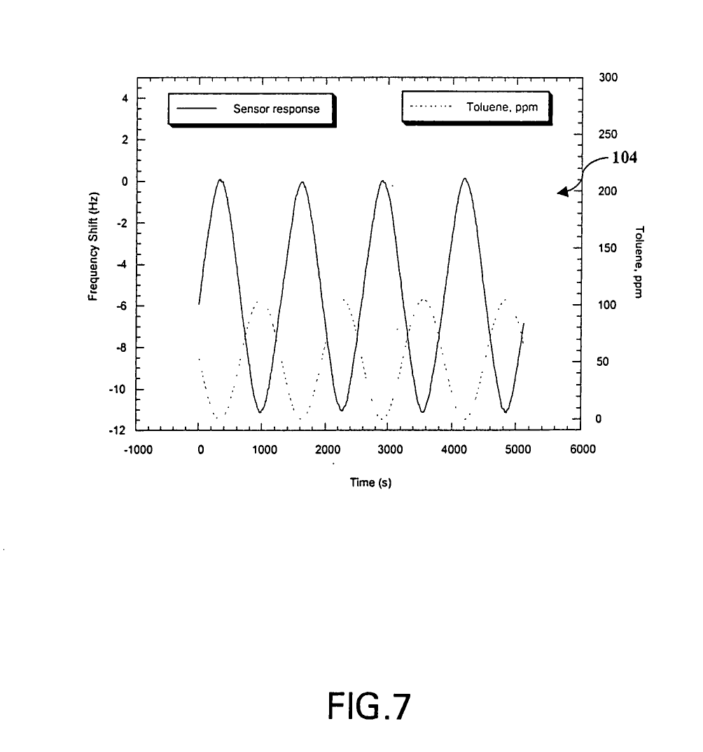

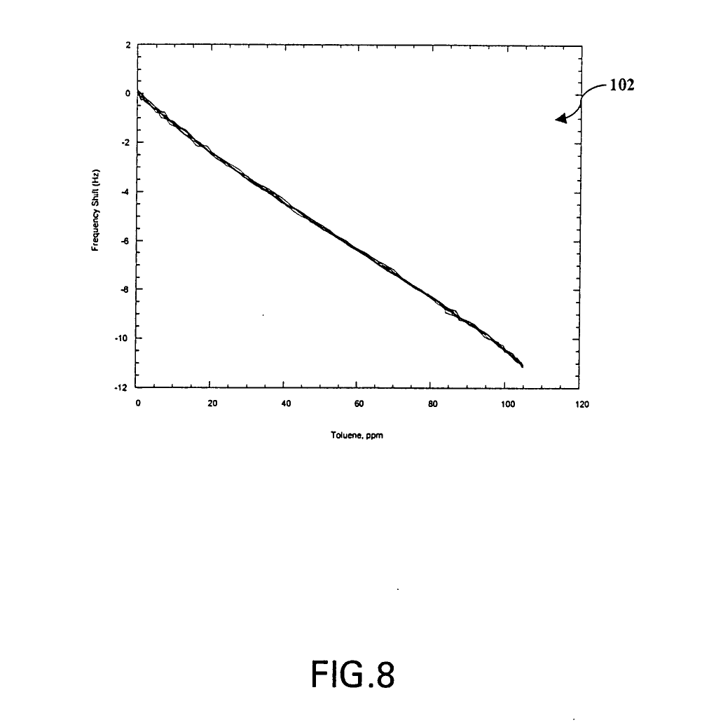

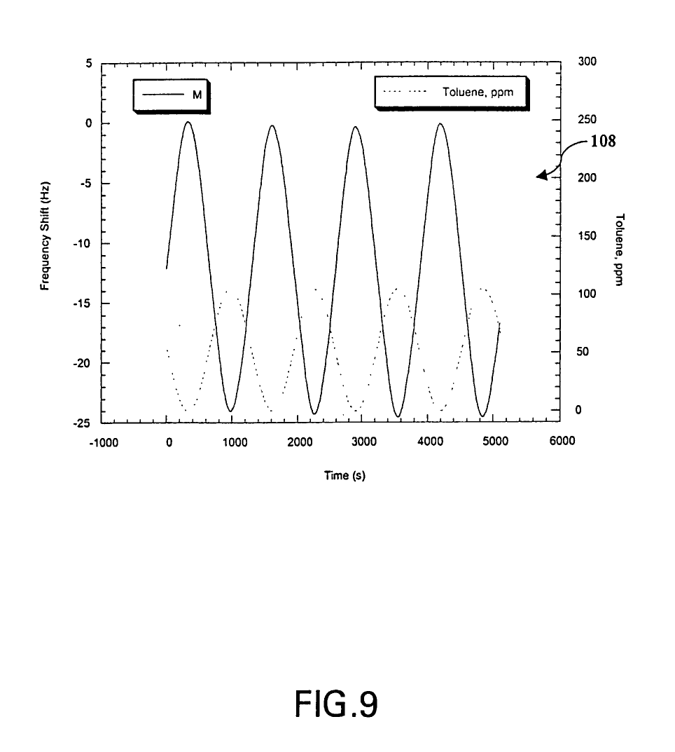

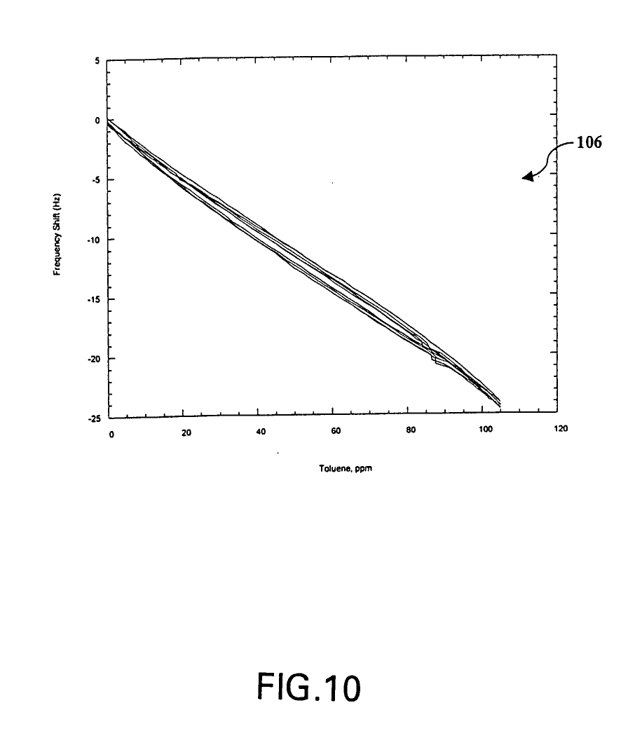

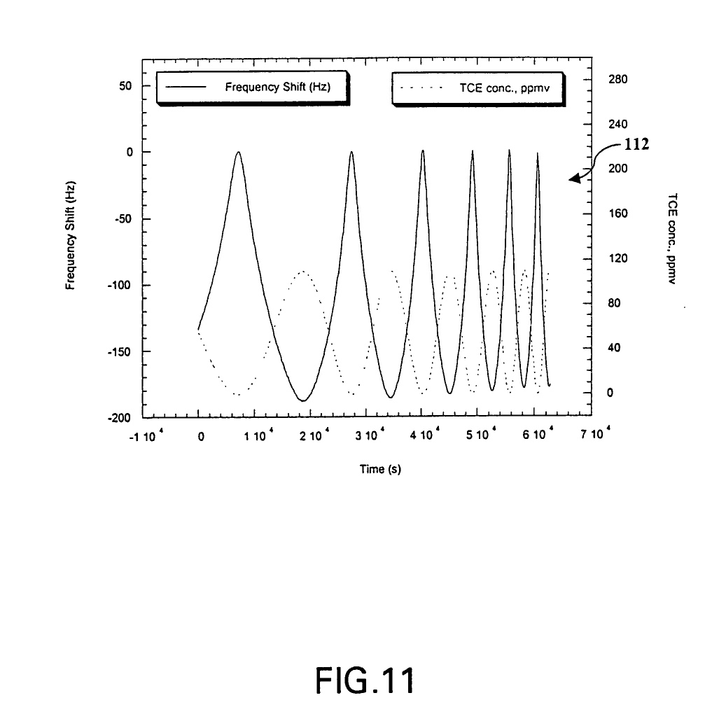

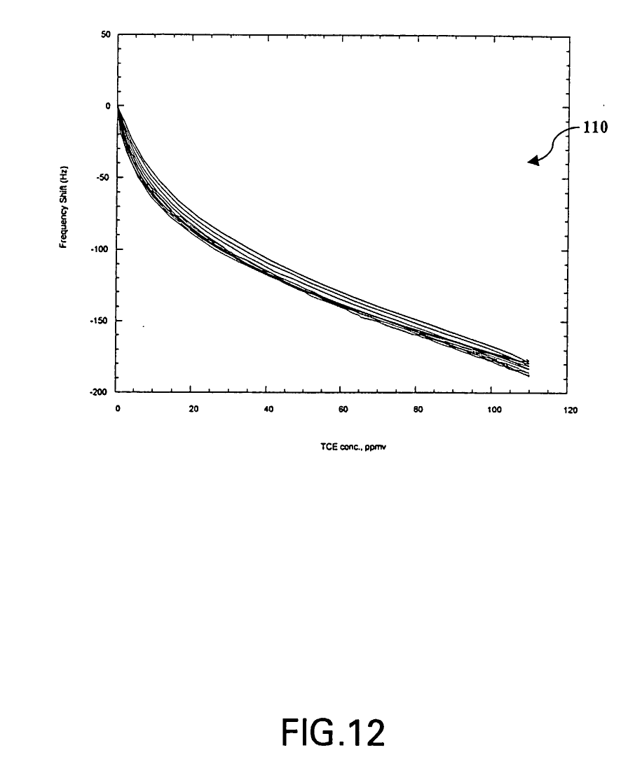

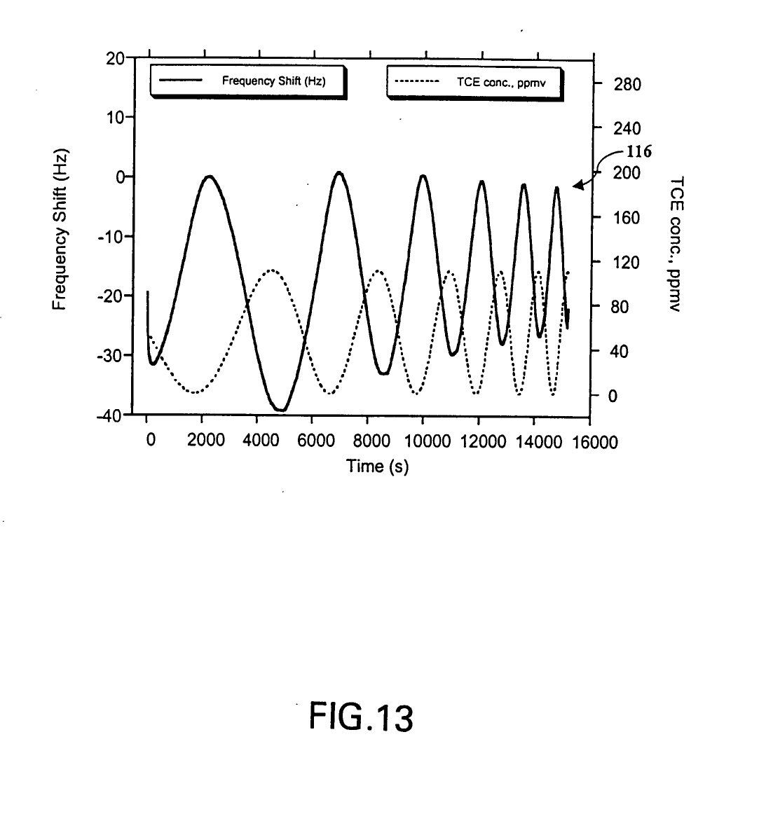

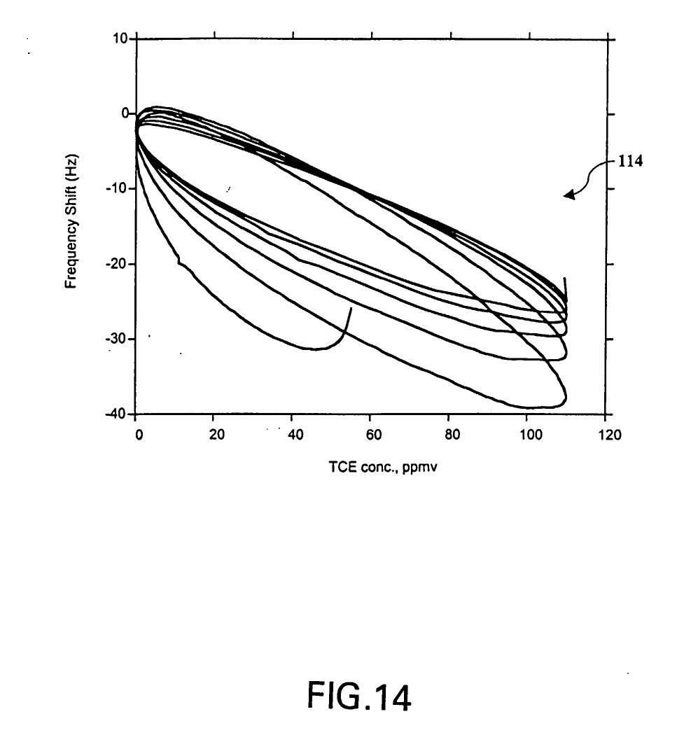

Sensor modulation was performed utilizing software written in LabVIEW. FIG. 6 illustrates a modulation sequence 100 of analyte concentration with a variable modulation cycle time. The signal change was recorded upon exposure of the TSM sensor array to varying concentrations of toluene or trichloroethylene (TCE) vapors. Sensor response was observed to be completely reversible. FIG. 7 illustrates the dynamic response 104 of a sensor coated with a Teflon AF film upon exposure to a sinusoidally-modulated concentration of toluene vapor from about 0 to about 105 ppm. FIG. 8 illustrates the response 102 of this sensor coated with a Teflon AF film upon such periodic exposure to toluene vapor. FIG. 9 illustrates the dynamic response 108 of a sensor coated with a poly(vinyl propionate) film upon exposure to a sinusoidally-modulated concentration of toluene vapor from about 0 to about 105 ppm. FIG. 10 illustrates the response 106 of this sensor coated with a poly(vinyl propionate) film upon such periodic exposure to toluene vapor. FIG. 11 illustrates the dynamic response 112 of a sensor coated with a Siltem 2000 film upon exposure to a sinusoidally-modulated concentration of TCE vapor from about 0 to about 110 ppm with a variable modulation cycle time. FIG. 12 illustrates the response 110 of this sensor coated with a Siltem 2000 film upon exposure to TCE vapor. The analyte concentration was sinusoidally-modulated with a variable modulation cycle time. FIG. 13 illustrates the dynamic response 116 of a sensor coated with an Ultem film upon exposure to a sinusoidally-modulated concentration of TCE vapor from about 0 to about 110 ppm with a variable modulation cycle time. FIG. 14 illustrates the response 114 of this sensor coated with an Ultem film upon exposure to TCE vapor. The analyte concentration was sinusoidally-modulated with a variable modulation cycle time.

This data demonstrates that periodic modulation of analyte concentration with a variable modulation cycle time provides an additional desired selectivity in sensor response. For example, upon reduction of the modulation cycle time, the amplitude in sensor response shown in FIG. 11 does not appreciably change while the amplitude in sensor response shown in FIG. 13 is reduced by more than 15 Hz as compared to the modulation depth of 40 Hz for the long modulation cycle. Another important selectivity parameter is the magnitude of the modulation amplitude change for the sorption and desorption portions of the sensor response cycle. As shown in FIG. 13, the modulation depth is reduced by about 15 Hz for the sorption portion of the modulation cycle. However, for the desorption portion of the modulation cycle, the reduction of the modulation amplitude is less than 5 Hz.

### WORKING EXAMPLE 3

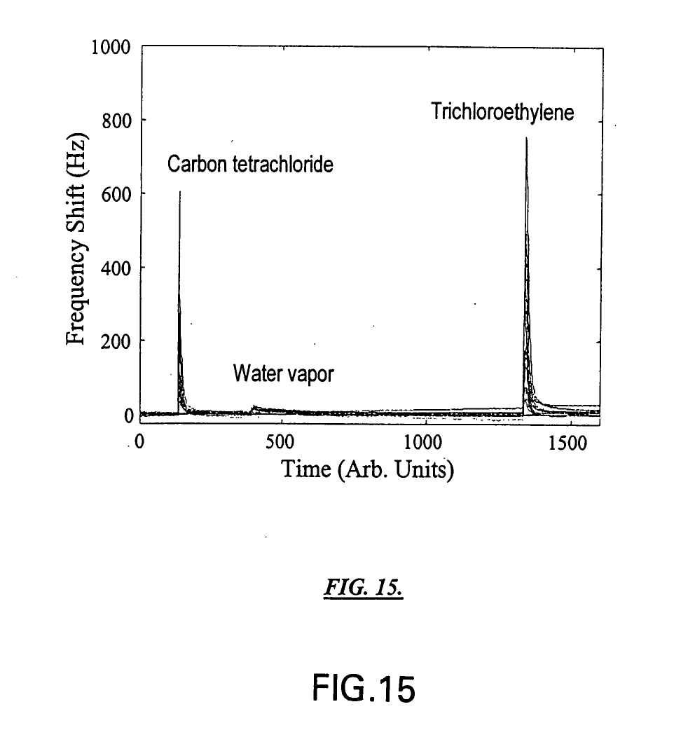

The simultaneous evaluation of multiple materials for their response to analytes was performed using a 24-channel array of acoustic wave devices or sensors. Each transducer was a 10-MHz AT-cut quartz crystal. Typical responses of the 24-channel system to short pulses of carbon tetrachloride vapor, water vapor, and TCE vapor is illustrated in FIG. 15. The vapor pulses were produced by introducing about 1 μL of solvent into a carrier-gas (nitrogen) delivery line. Rapid evaporation of the solvent was followed by the exponential dilution of the saturated vapor as indicated by a decrease in the signals of most of the channels. Several channels that did not produce measurable responses contained sealed crystals for the evaluation of the cross-talk and stability of the system.

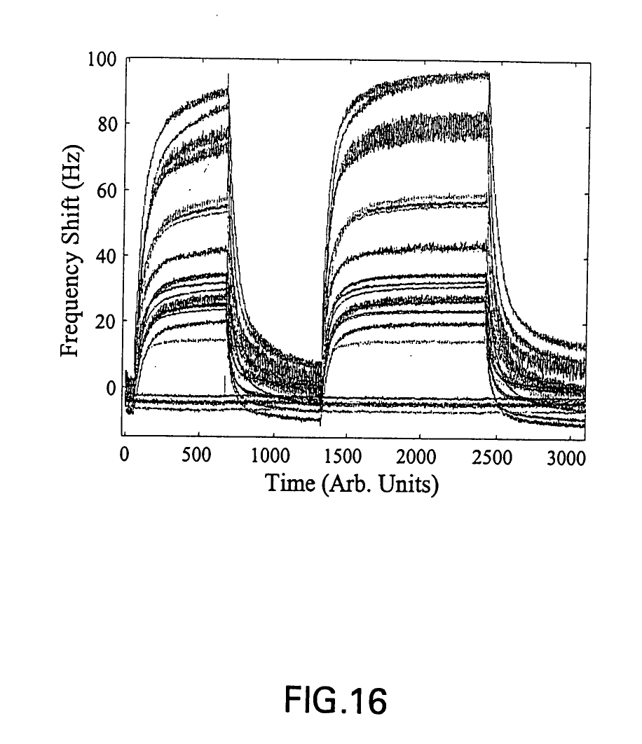

Selectivity and sensitivity of the sensor materials was evaluated using several gas mixtures, including tetrachloroethylene, TCE, vinyl chloride, and toluene. The concentrations of all of the gases were about 100 ppm in dry air. Response to the TCE was also evaluated with 1-ppm mixtures. FIG. 16 illustrates a typical response of the array to a blank gas (nitrogen) and 100 ppm of toluene. Several channels that did not produce measurable responses contained sealed crystals for the evaluation of the cross-talk and stability of the system. Other crystals were coated with different materials of variable thickness.

In each of the above embodiments, multivariate analysis may be utilized. Preferably, the multivariate analysis involves principal components analysis, neural networks analysis, partial least squares analysis, linear multivariate analysis, or nonlinear multivariate analysis. The present invention applies mathematical analysis in order to improve the resolution of the acoustic wave sensors, thus reducing the time required to identify and quantify fluids of interest, to improve the selectivity of the determinations of individual fluid components in multicomponent mixtures, and to improve the resolution of the acoustic wave sensors in characterizing unknown materials deposited onto the acoustic wave transducers. The mathematical analysis may involve multivariate analysis using multivariate measurement, wherein multivariate measurement includes measurements of more than one variable or response for each sample. For example, the velocity and the attenuation of an acoustic wave traveling through a deposited coating may be measured by a single acoustic wave transducer. In addition, different temporal characteristics of the measured transducer response may be used for multivariate analysis.

Suitable types of multivariate analysis include linear multivariate analysis, nonlinear multivariate analysis, partial least squares analysis, principal components analysis, and neural networks analysis. Generally, linear multivariate analysis is used to describe linear relationships between independent and dependent variables using straight line calibration functions, and non-linear multivariate analysis is used to describe nonlinear relationships between independent and dependent variables. Principal components analysis and factor analysis are transforms that find and interpret hidden complex and possibly causally determined relationships between features in a data set; the correlating features are then converted to factors which are themselves non-correlated. Partial least squares (PLS) analysis and principal components regression (PCR) analysis make use of an inverse calibration approach where it is possible to calibrate for the desired component(s) while implicitly modeling the other sources of variation; the difference between PLS and PCR is in how the factors are calculated. Neural networks analysis describes analysis of a data set to a specific problem by iterative adjustment of weights in a net during the learning process; this adaptation may be done either by comparison of the desired result with the data at the output of the net (supervised learning) or by maximizing differences in the learning data based on an arbitrary criterion of similarity (unsupervised learning). The multivariate analysis tools of the present invention improve the resolution of the acoustic wave sensors, thus reducing the time required to identify and quantify fluids of interest, improve the selectivity of determinations of individual fluid components in multicomponent mixtures, and improve the resolution of the acoustic wave sensors in characterizing unknown materials deposited onto the acoustic wave transducers.

Although the present invention has been described with reference to preferred embodiments and examples thereof, other embodiments may achieve the same results. Variations in and modifications to the present invention will be apparent to those of ordinary skill in the art and the following claims are intended to cover all such equivalents.

What is claimed is:

1. An apparatus for the rapid evaluation of a plurality of materials, the apparatus comprising:
a plurality of crystals operable for receiving an oscillating potential and oscillating, the plurality of crystals arranged in an array;
a plurality of oscillation devices operable for generating the oscillating potential, the plurality of oscillation devices arranged in an array;
means for measuring an output parameter each of the plurality of crystals;
means for setting a plurality of measurement parameters;
means for setting a plurality of control parameters; and
wherein the plurality of crystals are remotely coupled to the plurality of oscillation devices such that the plurality of crystals are exposed to a first operating environment and the plurality of oscillation devices are exposed to a second operating environment.

2. The apparatus of claim 1, wherein the plurality of measurement parameters comprise measurement parameters selected from the group consisting of total lines of data, number of points to average, trigger threshold, and integration time.

3. The apparatus of claim 1, wherein the plurality of control parameters comprise control parameters selected from the group consisting of flow rates of fluids delivered to the array of crystals, temperature, electromagnetic radiation, humidity, temporal profiles of fluid delivery, and temporal profiles of environmental conditions that affect the response of the crystals.

4. The apparatus of claim 1, further comprising one or more cables operable for coupling the plurality of crystals to the plurality of oscillation devices.

5. The apparatus of claim 1, further comprising one or more cables operable for remotely coupling the plurality of crystals to the plurality of oscillation devices.

6. The apparatus of claim 1, further comprising a time interval analyzer operable for determining when the means for measuring the output parameter for each of the plurality of crystals should measure the output parameter of each of the plurality of crystals.

7. The apparatus of claim 1, wherein the output parameter of each of the plurality of crystals comprises an output parameter selected from the group consisting of a fundamental oscillation frequency, a velocity of an acoustic wave, an attenuation of an acoustic wave, impedance phase and magnitude, conductance, and a capacitance change.

8. The apparatus of claim 1, wherein the means for measuring the output parameter each of the plurality of crystals measures the output parameter of each of the plurality of crystals in series.

9. The apparatus of claim 1, wherein the first operating environment and the second operating environment comprise predetermined temperature, pressure, chemical, and electromagnetic radiation environments.

10. The apparatus of claim 1, wherein the apparatus is used to detect material properties of interest, the material properties of interest comprising material properties selected from the group consisting of transition temperature, storage modulus, loss modulus, absorption, plasticization, crystallization, diffusion, permeation, barrier properties, physisorption, chemisorption, polymerization, and corrosion.

11. The apparatus of claim 1, wherein the apparatus is used to monitor film and particle deposition and removal from the plurality of crystals.

12. A method for enhancing the stability and the selectivity of each of a plurality of sensors of an array of sensors, the method comprising:
modulating each of the plurality of sensors of the array of sensors with respect to a predetermined parameter; and
wherein each of the plurality of sensors of the array of sensors comprises a material that is sensitive to a given environment such that when the sensor is exposed to the given environment a property of the material will change, measurably changing an output parameter of the sensor, and wherein the degree of change in the output parameter is correlated to the degree to which the given environment is present.

13. The method of claim 12, wherein each of the plurality of sensors of the array of sensors comprises a device selected from the group consisting of a thickness-shear mode (TSM) device, a surface acoustic wave (SAW) device, an acoustic plate mode (APM) device, a flexural plate wave (FPW) device, and a surface transverse wave (STW) device.

14. The method of claim 12, wherein the given environment comprises an environment selected from the group consisting of a temperature environment, a pressure environment, a chemical environment, and an electromagnetic radiation environment.

15. The method of claim 12, wherein the property of the material comprises the mass of the material.

16. The method of claim 12, wherein the property of the material comprises a viscoelastic property of the material.

17. The method of claim 12, wherein the output parameter of the sensor comprises an output parameter selected from the group consisting of a fundamental oscillation frequency, a velocity of an acoustic wave, an attenuation of an acoustic wave, impedance phase and magnitude, conductance, and a capacitance change.

18. The method of claim 12, wherein modulating each of the plurality of sensors of the array of sensors with respect to a predetermined parameter comprises modulating each of the plurality of sensors of the array of sensors with respect to sample flow rate.

19. The method of claim 12, wherein modulating each of the plurality of sensors of the array of sensors with respect to a predetermined parameter comprises modulating each of the plurality of sensors of the array of sensors with respect to sensor temperature.

20. The method of claim 12, wherein modulating each of the plurality of sensors of the array of sensors with respect to a predetermined parameter comprises modulating each of the plurality of sensors of the array of sensors with respect to a predetermined parameter utilizing a predetermined function.

21. The method of claim 20, where the predetermined function comprises a function selected from the group consisting of a step function, a square function, a saw function, and a sinusoidal function.

22. The method of claim 12, wherein modulating each of the plurality of sensors of the array of sensors with respect to a predetermined parameter comprises modulating each of the plurality of sensors of the array of sensors with respect to a predetermined parameter comprising a parameter selected from the group consisting of a change in the modulation period, modulation duty cycle, modulation waveform, modulation depth, and a modulation phase between several modulated parameters.

23. The method of claim 12, wherein modulating each of the plurality of sensors of the array of sensors with respect to a predetermined parameter comprises modulating each of the plurality of sensors of the array of sensors with respect to at least two predetermined parameters utilizing a predetermined function wherein the phase difference between the at least two predetermined parameters is changing as a function of time.

24. The method of claim 12, further comprising exposing a gas flow disposed around each of the plurality of sensors of the array of sensors to an analyte-removing source, the analyte-removing source creating a cleaned gas flow.

25. The method of claim 22, wherein the analyte-removing source comprises a corona discharge.

26. A method for the rapid evaluation of the extractability of materials from a plurality of samples, the method comprising:
providing a plurality of samples;
disposing the plurality of samples in a plurality of solvents for a first predetermined period of time;
providing a plurality of acoustic wave devices;
measuring a predetermined output parameter of each of the plurality of acoustic wave devices;
disposing the plurality of acoustic wave devices in the plurality of solvents for a second predetermined period of time;
removing the plurality of acoustic wave devices from the plurality of solvents;
measuring the predetermined output parameter of each of the plurality of acoustic wave devices; and
correlating the change in the predetermined output parameter of each of the plurality of acoustic wave devices to the extractability of materials from the plurality of samples.

27. The method of claim 26, further comprising arranging the plurality of samples in an array.

28. The method of claim 26, further comprising arranging the plurality of solvents in a array.

29. The method of claim 26, further comprising arranging the plurality of acoustic wave devices in an array.

30. The method of claim 26, wherein removing the plurality of acoustic wave devices from the plurality of solvents comprises evaporating the plurality of solvents;

31. The method of claim 26, wherein providing the plurality of acoustic wave devices comprises providing a plurality of acoustic wave devices selected from the group consisting of thickness-shear mode (TSM) devices, surface acoustic wave (SAW) devices, acoustic plate mode (APM) devices, flexural plate wave (FPW) devices, and surface transverse wave (STW) devices.

32. The method of claim 26, wherein measuring the predetermined output parameter of each of the plurality of acoustic wave devices comprises measuring an output parameter selected from the group consisting of a fundamental oscillation frequency, a velocity of an acoustic wave, an attenuation of an acoustic wave, impedance phase and magnitude, conductance, and a capacitance change.

33. The method of claim 26, wherein measuring the predetermined output parameter of each of the plurality of acoustic wave devices comprises measuring the predetermined output parameter of each of the plurality of acoustic wave devices simultaneously.

34. The method of claim 26, wherein the plurality of samples comprise a plurality of combinatorially-developed materials.

35. The method of claim 26, wherein the materials comprise low-molecular weight materials.

36. The method of claim 26, wherein the plurality of solvents comprise solvents selected from the group consisting of water, fuels, alkaline and acidic solutions, and organic solvents of different polarities.

37. The method of claim 26, further comprising identifying an extractable utilizing a mathematical analysis tool comprising a multivariate analysis tool selected from the group consisting of principal components analysis, neural networks analysis, partial least squares analysis, linear multivariate analysis, and nonlinear multivariate analysis.

38. A method for the rapid evaluation of the extractability of materials from a plurality of samples, the method comprising:
providing a plurality of samples, the plurality of samples arranged in an array;
disposing the plurality of samples in a plurality of solvents for a first predetermined period of time, the plurality of solvents arranged in an array;
providing a plurality of acoustic wave devices, the plurality of acoustic wave devices arranged in an array;
simultaneously measuring a fundamental oscillation frequency of each of the plurality of acoustic wave devices;
disposing the plurality of acoustic wave devices in the plurality of solvents for a second predetermined period of time;
evaporating the plurality of solvents;
simultaneously measuring the fundamental oscillation frequency of each of the plurality of acoustic wave devices; and
correlating the change in the fundamental oscillation frequency of each of the plurality of acoustic wave devices to the extractability of materials from the plurality of samples.

39. The method of claim 38, wherein providing the plurality of acoustic wave devices comprises providing a plurality of acoustic wave devices selected from the group consisting of thickness-shear mode (TSM) devices, surface acoustic wave (SAW) devices, acoustic plate mode (APM) devices, flexural plate wave (FPW) devices, and surface transverse wave (STW) devices.

40. The method of claim 38, wherein the materials comprise low-molecular weight material fractions.

41. The method of claim 38, wherein the plurality of solvents comprise solvents selected from the group consisting of water, fuels, alkaline and acidic solutions, and organic solvents of different polarities.

42. An apparatus for the rapid evaluation of the extractability of materials from a plurality of samples, the apparatus comprising:
a plurality of wells, the plurality of wells arranged in an array;
a plurality of samples and a plurality of solvents disposed within the plurality of wells, the plurality of samples and the plurality of solvents combining to form a plurality of solutions containing the materials;
a plurality of acoustic wave devices, wherein each of the plurality of acoustic wave devices comprises at least one surface operable for attracting at least one of the materials from the plurality of solutions, the plurality of acoustic wave devices arranged in an array;
means for measuring a predetermined output parameter of each of the plurality of acoustic wave devices; and
a correlation factor operable for correlating a change in the predetermined output parameter of each of the plurality of acoustic wave devices to the extractability of the materials from the plurality of samples.

43. The apparatus of claim 42, wherein the plurality of acoustic wave devices comprise a plurality of acoustic wave devices selected from the group consisting of thickness-shear mode (TSM) devices, surface acoustic wave (SAW) devices, acoustic plate mode (APM) devices, flexural plate wave (FPW) devices, and surface transverse wave (STW) devices.

44. The apparatus of claim 42, wherein the predetermined output parameter of each of the plurality of acoustic wave devices comprises an output parameter selected from the group consisting of a fundamental oscillation frequency, a velocity of an acoustic wave, an attenuation of an acoustic wave, impedance phase and magnitude, conductance, and a capacitance change.

45. The apparatus of claim 42, wherein the means for measuring the predetermined output parameter of each of the plurality of acoustic wave devices are operable for measuring the predetermined output parameter of each of the plurality of acoustic wave devices simultaneously.

46. The apparatus of claim 42, wherein the plurality of samples comprise a plurality of combinatorially-developed materials.

47. The apparatus of claim 42, wherein the plurality of materials comprise additives selected from the group consisting of low-molecular weight material fractions.

48. The apparatus of claim 42, wherein the plurality of solvents comprise solvents selected from the group consisting of water, fuels, alkaline and acidic solutions, and organic solvents of different polarities.

49. The apparatus of claim 42, further comprising a plurality of electrodes coupled to the plurality of acoustic wave devices.

50. An apparatus for the rapid evaluation of chemical sensitivity properties of a plurality of materials, the apparatus comprising:
a plurality of crystals coated with materials of interest and operable for receiving an oscillating potential and oscillating, the plurality of crystals arranged in an array;
a plurality of oscillation devices operable for generating the oscillating potential, the plurality of oscillation devices arranged in an array;
means for measuring an output parameter of each of the plurality of crystals;
means for setting a plurality of measurement parameters;
means for setting a plurality of control parameters; and
wherein the plurality of crystals is remotely coupled to the plurality of oscillation devices such that the plurality of crystals are exposed to a first operating environment and the plurality of oscillation devices are exposed to a second operating environment.

51. The apparatus of claim 50, wherein the plurality of measurement parameters comprise measurement parameters selected from the group consisting of total lines of data, number of points to average, trigger threshold, and integration time.

52. The apparatus of claim 50, wherein the plurality of control parameters comprise control parameters selected from the group consisting of flow rates of fluids delivered to the array of crystals, temperature, electromagnetic radiation, humidity, temporal profiles of fluid delivery, and temporal profiles of environmental conditions that affect the response of the crystals.

53. The apparatus of claim 50, wherein the output parameter of each of the plurality of crystals comprises an output parameter selected from the group consisting of a fundamental oscillation frequency, a velocity of an acoustic wave, an attenuation of an acoustic wave, impedance phase and magnitude, conductance, and a capacitance change.

54. The apparatus of claim 50, wherein the evaluation process of the chemical sensitivity properties is controlled automatically by a computer, wherein the computer commands desired concentrations of fluids to be delivered to the array of coated crystals.
